(19) United States
(12) Patent Application Publication
(10) Pub. No.: US 2026/0286264 A1
(43) Pub. Date: Sep. 24, 2026

(54) METHOD AND APPARATUS FOR CULTIVATING AT LEAST ONE CELL IN A CELL CULTURE PLATE

(71) Applicant: DEMCON SYNC BIOSYSTEMS B.V., ENSCHEDE (NL)

(72) Inventors: Jasmijn BÜSKENS, ENSCHEDE (NL); Berend Jan VAN MEER, ENSCHEDE (NL); Mike SMIT, ENSCHEDE (NL); Peter Paul VAN SWINDEREN, ENSCHEDE (NL); Tessa DE KORTE, ENSCHEDE (NL)

(73) Assignee: DEMCON SYNC BIOSYSTEMS B.V., ENSCHEDE (NL)

(21) Appl. No.: 19/473,967

(22) PCT Filed: Apr. 11, 2024
(86) PCT No.: PCT/NL2024/050187

§ 371 Date: Oct. 9, 2025

(30) Foreign Application Priority Data
Apr. 11, 2023 (NL) 2034536

(51) Int. Cl. C12M 1/32; C12M 1/00; C12M 3/00

(52) U.S. Cl. CPC C12M 23/12; C12M 23/40; C12M 23/48; C12M 29/00; C12M 29/06

(57) ABSTRACT

A method for cultivating at least one cell in a cell culture plate, wherein the method comprises the steps of: providing a cell culture plate comprising a least one well containing at least one cell and a first fluid, for instance a cell culture medium; providing a second fluid, for instance a cell culture medium, in a fluid container; providing a fluid manifold comprising a fluid outlet extending in the well and coupled to the fluid container and a fluid inlet arranged to receive fluid from said well and coupled to an fluid output; and replacing the fluid in said well by introducing, from said fluid outlet, said second fluid in said well while removing, using said fluid inlet, fluid from said well until the well is homogenously filled with the second fluid.

The present invention relates to a method and apparatus for cultivating one or more cells in a cell culture plate. The invention further relates to a manifold for use in such a method or apparatus.

Cells or cell cultures held in wells of for instance cell culture plate are widely used in the pharmaceutical research. Cells are exposed to environments mimicking the conditions within a patient. There is however a mismatch in translation of in vitro to in vivo which limits the predictability of effects in patients, leading to inefficiency and higher drug prizes, potentially missing effective therapies and resulting in a high number of animals used.

More complex models, such as micro physiological systems, are being introduced to solve these issues but have their limitations. Organoids allow more complex biology, but in the same simplistic well plates, while Organ-on-Chip allows complex and controlled environment but require cells to be cultured in a new, non-standard device. This typically requires a 3-12 months new protocol development, which hampers adoption.

It is a goal of the present invention, next to other goals, to provide an improved method for cultivating at least one cell in a cell culture plate, which preferably allows a more complex environment to existing models, while preferably keeping the threshold low by not changing the cell culture protocols.

This goal, amongst other goals, is met by a method for cultivating at least one cell, such as a cell culture, in for instance a cell culture plate, wherein the method comprises the steps of:
providing a cell culture plate comprising at least one well containing at least one cell and a first fluid, for instance a cell culture medium;
providing a second fluid, for instance a cell culture medium, in a fluid container;
providing a fluid manifold comprising a fluid outlet extending in the well and coupled to the fluid container and a fluid inlet arranged to receive fluid from said well and coupled to a fluid output;
replacing the fluid in said well by introducing, from said fluid outlet, said second fluid in said well while removing, using said fluid inlet, fluid from said well until the well is homogenously filled with the second fluid.

Replacing the fluid in a well, which may be a cell culture medium, by a second fluid, which preferably has predefined and conditioned properties, allows subjecting the at least one cell to this same predefined conditions.

The second fluid with preferably predefined conditions now replaces the fluid in the well, instead of adding a component, for instance an active ingredient, to the fluid in the well to change the conditions in said well towards a desired condition of said component in the well. This is less accurate and may take longer, as these known systems typically rely on slow diffusion for distribution. That is, it is more difficult to for instance achieve a desired homogeneous concentration with predefined conditions in the well in a short amount of time by adding a component to the well than by replacing the fluid in the well with fluid having said predefined concentration.

The step of providing the second fluid may comprise conditioning the second fluid, for instance by adding a predefined amount of components to a predefined amount of fluid, possibly including a mixing step, in order to provide the second fluid having predefined properties suitable for a pharmacokinetic model. The second fluid may be in a fluid container.

The fluid manifold is arranged to couple to a well and is arranged to replace the fluid in a well with the second fluid. The manifold hereto has an outlet for introducing fluid in the well. Preferably, and as will be discussed in greater detail below, the outlet extends in the well and may for instance comprise an outlet tube arranged to introduce the fluid in the well. The outlet of the manifold is coupled, for instance using suitable tubing, to a source of fluid, for instance a fluid container as mentioned above.

For removing the fluid from the well, a fluid inlet is provided. The fluid inlet of the manifold may then be coupled to a fluid output, for instance a reservoir for storing used fluid. The fluid as removed or received from the well may be analysed.

The fluid is preferably replaced in the well by introducing fluid in the well using the outlet of the manifold, while at the same time fluid is removed or received from the well using the fluid manifold inlet. The step of replacing the fluid may be stopped when the first fluid is homogeneously replaced by the second fluid, or at least substantially.

In the context of this disclosure, the term homogeneously filled is to be understood that the first, initial fluid in the well is substantially completely replaced by the second fluid, at least at the location of the cell(s) in the well. At least the fluid in (direct) contact with, or generally surrounding, the cell(s) is then preferably homogenously replaced by the second fluid. Preferably, a share of first fluid after the step of replacing the fluid, at least at the location of the cell(s) is the well, is less than 5 vol. % of the well content, more preferably less than 3 vol %, even more preferably less than 2.5 vol. % of the well and even more preferably less than 1 vol. % of the well.

The method as described is particularly suitable for replacing a fluid in a well in the context of a method for cultivating at least one cell in a cell culture plate. The method, and manifold and apparatus as described further below, may also be generally used to replace fluid, or even introduce fluid, in a well, or generally in a container, even without at least one cell.

According to a preferred embodiment, the step of replacing the fluid comprises maintaining a substantial constant fluid level in said well. This ensures that the at least one cell, for instance a cell culture or organoid, in the well is submersed in fluid at all times. Alternatively, it is preferred that the at least one cell in the well is kept submersed or at least wet. Preferably, and as explained in further detail below, the step of replacing the fluid comprises displacing the first fluid by the second fluid. The step of removing then preferably comprising inducing an overflow of the first fluid into for instance an overflow reservoir and removing the fluid from the overflow reservoir. As such, a substantial fluid level in the well can be maintained.

According to a further preferred embodiment, the step of introducing the second fluid comprising introducing said second fluid at a sufficiently low flow rate and/or pressure to prevent disturbing the at least one cell. The fluid is preferably introduced, and removed as will be explained in greater detail below, at a sufficiently low rate and/or pressure such that the at least one cell is not, or substantially not, damaged or even disturbed to ensure optimal cell culture conditions. More preferably, introducing and/or removing fluid takes place such that the integrity and/or confluency of a cell culture in the well is not disturbed, at least not significantly disturbed. For example, the force of liquid flowing over the surface of the cells can create shear stress, which can cause damage to the cell membrane and affect cell viability and function. This can be particularly problematic for delicate cell types or for cultures that are sensitive to mechanical stress. Furthermore, when media is added to a cell culture it can create turbulence and disturbance in the culture, which can affect the distribution of cells and cell aggregates within the culture. This can lead to uneven growth, changes in cell morphology, and disruptions to cellular interactions that are critical for maintaining a healthy and functional cell culture.

It is further noted that next to the advantages in terms of the prevention of disturbance of the cells, the method further allows replacing the fluid in reproduceable and constant manner, i.e., according to predefined conditions, for instance when compared to replacing the fluid using pipetting techniques, in particular manual pipetting.

Generally, replacing the fluid in the well preferably takes place in conditions similar to the conditions in a typical bioreactor as known in the art. Models which are readily available for these bioreactors can then be applied in the method of the current disclosure to reduce mechanical stress on the cultivated cells, such as stirred-tank bioreactors designed with low-shear impellers, wave bioreactors that use a wave-like motion to gently mix the cell culture, or rotating wall vessel bioreactors which use a rotating vessel to create a microgravity environment for cell culture, thereby reducing the effects of sedimentation and buoyancy-driven convection that can create shear stress on the cells. As in the method of present invention, the above bioreactors also create a more homogeneous distribution of the cell culture media comprised of nutrients (and/or drug) and oxygen throughout the culture.

Preferably and to maintain the integrity of the at least one cell, the step of introducing the second fluid comprising introducing said second fluid at a sufficiently low flow rate and/or pressure to exert a shear stress on said at least one cell lower than 0.1 Pa, preferably lower than 0.05 Pa, more preferably lower than 0.01 Pa.

According to a further preferred embodiment, the step of introducing the second fluid comprising introducing said second fluid at a flow rate of between 0.2 and 1.4 ml/min, preferably between 0.8 and 1.2 ml/min, more preferably between 0.95 and 1.05 ml/min.

According to a further preferred embodiment, the step of replacing the fluid, at least 95 vol. % of said fluid, takes within 5 minutes, preferably within 2 minutes, more preferably within 1.5 minutes and most preferably within 1 minute. This timeframe allows subjecting a cell, or cell culture, to a dynamic pharmacokinetic profile.

According to a further preferred embodiment, the step of introducing the second fluid comprises introducing said second fluid from a nozzle of said outlet from a location below the fluid level in said well. The outlet, for instance in the form of an outlet tube, then preferably extends in the well wherein the outlet tube partly extends in the fluid, at least an outlet side thereof. Introducing the fluid form below the fluid level in the well allows efficient displacement of the first fluid by the second fluid, while preventing disturbance of the at least one cell.

The method is particularly suitable to be used in combination with a cell culture plate having at least one well. Typically, such a plate comprises a plurality of wells arranged in an array. A well typically has a depth of approximately 11 mm (10.9 mm) and a diameter of approximately 7 mm (6.96 mm), giving a typical absolute volume of approximately 400 ul (382 ul). Typically, the working volume varies between 36-340 ul.

Preferably, the second fluid is introduced from said nozzle extending at a distance of 0.1-5 mm, preferably between 1-4 mm, more preferably between 2-3 mm, from a bottom of said well.

To improve the distribution of the second fluid upon introduction in the well, the nozzle preferably comprises a fluid channel widening towards the outlet side of the outlet. The widening channel not only distributes the second fluid over a larger volume in the well, but also serves to lower the pressure of the second fluid due to the increased cross-sectional area of the channel. This further prevents disturbance of the cell(s).

To further enhance the distribution of the second fluid in the well upon introduction, preferably the wall of the widening channel is substantially aligned with the corner between the bottom and the side wall of the well. Fluid is then preferably urged towards the (complete) bottom of the well, urging the first fluid upwardly. At that location, the fluid in the well can be removed, for instance using an overflow technique as will be explained in greater detail below.

According to a further preferred embodiment, the step of replacing the fluid comprises introducing the second fluid in the in well and removing the fluid at a second location, not in fluid communication with the well. A direct fluid connection between the outlet and the inlet is then prevented, which improves the efficiency of the replacement of the fluid in the well, while it preferably also prevents backflow. In use, the location of the removal of fluid is then not a communicating vessel with the location of introduction. Preferably, there is provided an air lock between the inlet location and the outlet location.

It will be appreciated that although the concept of replacing the fluid at locations not in fluid communication, also as further described below, is particularly useful to efficiently replace the fluid homogeneously, this concept may also be applied in a method for generally replacing fluid or even more generally for adding a fluid to a well.

According to a further preferred embodiment, the step of removing fluid from the well comprises receiving overflow fluid from said well in an overflow reservoir and removing, using said fluid inlet, fluid from said overflow reservoir. Fluid is then not directly removed from the well, but from an overflow reservoir. Direct suction in the well is then prevented, which improves the replacement process of the fluid by the second fluid. The overflow reservoir preferably extends above the well in use. Fluid is urged upwardly, by introducing the second fluid, and is received in the overflow reservoir. The reservoir may then comprise a fluid suction for removing fluid from said reservoir, preferably while preventing back flow, more preferably by providing an air lock, between the overflow reservoir and the fluid in the well.

It is preferred when the step of introducing the second fluid comprises urging the fluid in the well upwardly towards the overflow reservoir. The fluid is then efficiently replaced.

A further preferred embodiment further comprises the steps of providing a third fluid and replacing the second fluid in said well by introducing, from said fluid outlet, said third fluid in said well. Preferably, introducing takes place while removing, using said fluid inlet, fluid from said well until the well is homogenously filled with the third fluid. A separate container may be provided to supply the third fluid. Additionally or alternatively, the third fluid may be prepared in the container which previously held the second fluid.

As mentioned above, a cell culture plate typically comprises a plurality of wells. The method is then preferably arranged to replace the fluid in a plurality of wells. Although it is possible to provide a suitable manifold for each well, it is preferred if the manifold is arranged to couple to a plurality of wells and more preferably to replace the fluid in a plurality of cells, preferably simultaneously. The manifold may then be coupled, for instance using suitable tubing, to a supply of second fluid, for instance the fluid container as mentioned above.

Therefore, a further preferred embodiment relates to a method for cultivating a plurality of at least one cell in a plurality of wells in a cell culture plate, wherein:
the step of providing the cell culture plate comprises providing a cell culture plate comprising a plurality of wells, wherein a plurality of said wells contain at least one cell and a fluid such as a cell culture medium;
the step of providing the fluid manifold comprises providing a fluid manifold comprising a plurality of fluid outlets extending in respective wells and coupled to a fluid container containing the second fluid.
replacing the fluid in at least one of said wells by introducing, from at least one fluid outlet, said second fluid in said well while removing, using said fluid inlet, fluid from said well until the well is homogenously filled with the second fluid.

Preferably, the step of replacing the fluid comprises introducing, from a plurality of fluid outlets, said second fluid in a plurality of wells while removing, using said fluid inlet, fluid from said wells until the wells are homogenously filled with the second fluid. The fluid of a plurality of wells is then replaced simultaneously.

More preferably, wherein the step of removing fluid from the well comprises receiving overflow fluid from a plurality of wells in an overflow reservoir and removing, using said fluid inlet, fluid from said overflow reservoir. A manifold preferably comprises an overflow reservoir common to a plurality of wells. That is, excess fluid, urged from the well due to the introduction of the second fluid, of a plurality of wells is then received in a common overflow reservoir and can be removed from said overflow reservoir using the outlet, for instance in the form of a suction device.

A further preferred embodiment further comprises the step of analysing the at least one cell using a microscope. The behaviour of the cells to the different fluids can then be determined and studied.

A further preferred embodiment further comprises the step of analysing the fluid from the well, for instance the fluid received in the fluid output. The step of analysing may for instance comprise determining the presence of any reactants in the fluid, for instance removed from the well. Analysing the fluid which is removed from the well has the advantage that no measurement on the fluid in the well need to take place, thereby preventing the disturbance of the cell(s).

According to a further aspect, a fluid manifold is provided, in particular for use in a method as described above, wherein the manifold is arranged to be coupled to a cell culture well, for instance of a cell culture plate comprising a plurality of cell culture wells, wherein the fluid manifold comprises:
a fluid outlet comprising an outlet tube arranged to introduce fluid in a well at a first location, wherein the fluid outlet is coupled to a fluid outlet connector;
a fluid inlet arranged to receive fluid from said well at a second location, wherein said fluid inlet is coupled to a fluid inlet connector.

Preferably, the fluid manifold is arranged to couple to said well such that said first location of introducing the fluid is inside said well and said second location for receiving the fluid is outside the well, wherein in use the first and second locations are not in fluid communication. As mentioned above, a manifold, or generally a fluid distributing device, providing a location for the introduction of fluid which is in use not fluidly coupled to the location of the removal of fluid allows an efficient replacement of fluid in a well. A direct flow of fluid from an outlet to an inlet is then prevented. More preferably, an air lock is provided between the first and second locations.

The outlet tube preferably comprises a fluid channel for transporting the fluid to an outlet side of the outlet tube. The channel may have a substantially constant cross section. Preferably, the outlet tube however comprises a nozzle formed by a fluid channel widening towards the outlet side of the outlet tube. This improves the distribution of fluid in the well, while also reducing the fluid pressure.

More preferably, the fluid manifold is arranged to couple to said well such that the wall of the widening channel is substantially aligned with the corner between the bottom and the side wall of the well. In use, that is in a combination of a well and the manifold, the widening wall, which may have a frustoconical shape is then aligned with the corner of the well. In other words, a virtual extension of the widening wall will cross, at least approximately, at the location of the corner.

According to a further preferred embodiment, the manifold comprises a plurality of outlet tubes, each arranged for introducing fluid in a respective well, wherein the plurality of outlet tubes is preferably coupled to a common fluid outlet connector. Using a single coupling to for instance a container as fluid source, the fluid can be distributed to a plurality of wells.

Preferably, the plurality of outlet tubes is coupled to the common fluid outlet connector using respective flow resistors. The outlet tubes then each introduce fluid at the same pressure and preferably flow rate.

Preferably, the outlet tube is arranged to be centred in the well, in particular when the manifold is placed on a wells plate. This causes the second fluid introduced in the well by the outlet tube to uniformly displace the first fluid in the well, preventing turbulence and minimizing mixing between the first and second fluid.

It will be appreciated that the concept of the aligned wall or the plurality of tubes generally may also be employed in a manifold the first and second locations in fluid communication. Also, the concept of the first and second location not being in fluid communication may likewise be employed in a manifold, or generally fluid distributing device, having general outlets, not necessarily tubes.

Preferably, the manifold comprises a passage for receiving fluid from the well. In a preferred embodiment, the passage extends substantially coaxially to the outlet tube. Preferably, the passage comprises one or more openings extending around the outlet tube. In a preferred embodiment, the passage comprises a single opening extending around the outlet tube. In another embodiment, the passage comprises a plurality of openings, extending around the outlet tube. Preferably, the one or more passages extend in a substantially rotationally, or radially, symmetrical manner around the outlet tube. The passage, comprising one or more openings, extending around the outlet tube allows for a uniform pressure distribution in the fluid flowing out of the well, reducing turbulence in the fluid and increasing homogeneity.

Preventing turbulence and minimizing mixing during fluid replacement increases the gradient of the transition from the first fluid being replaced by the second fluid. In other words, reducing mixing between the first and second fluid during the replacement of the first fluid by the second fluid in the well minimizes the period wherein the well contains an undetermined environment, as the specific composition of a mixture of an unknown ratio between the first and second fluid is unknown.

A further preferred embodiment further comprises an overflow reservoir for receiving fluid from said well, wherein the fluid inlet is arranged to remove fluid from the overflow reservoir. Fluid is preferably pushed out of the well by introducing the second fluid, thereby urging the fluid into the overflow reservoir. Preferably, the outlet tube is arranged for introducing fluid from a lower outlet side thereof, wherein the overflow reservoir extends at a location above the lower outlet side of the outlet tube. The overflow reservoir is preferably located above a well, in coupled state.

More preferably, the overflow reservoir comprises an outer wall and an inner wall, preferably a cylindrical inner wall, defining the reservoir therebetween, wherein the outlet tube of the outlet extends inside the inner wall at a distance from said inner wall for defining a passage for fluid from the well into the overflow reservoir. A passage is thus defined from the well into the overflow reservoir. Preferably, manifold is arranged such that an air portion is maintained between the well and the fluid reservoir. More preferably, this passage contains an airlock, ensuring that first and second locations are not fluidly connected.

Preferably, the outlet tube is centred in the cylindrical inner wall. Thereby, the passage for fluid from the well into the overflow reservoir extends in a radially, or rotationally, symmetrical manner around the outlet tube. This allows for a uniform pressure distribution in the fluid being pushed out of the well through the passage, preventing turbulence and improving homogeneity.

Preferably, when the manifold is placed on a wells plate, the cylindrical inner wall of a passage is aligned with the wall of the well. This provides a smooth and flush transition between the wall of the well and the wall of the passage, allowing for an even more uniform pressure distribution in the fluid being pushed out of the well, through the passage, further improving homogeneity. As a consequence of the wall of the passage being aligned with the wall of the well, and the outlet tube being centred in the cylindrical inner wall of the passage, the outlet tube is centred in the well.

Preferably, the fluid reservoir and the passage for fluid are open to the environment of the fluid manifold. The passage further allows the outside atmosphere, that the atmosphere wherein the manifold extends, which may be a closed and conditioned atmosphere, to come into contact with the well. This is beneficial for the cell(s) in the well and similar to standard cell culture in incubators.

When the fluid manifold comprises a plurality of outlet tubes, the overflow reservoir preferably comprises a plurality of cylindrical inner walls, wherein each of the plurality of outlet tubes extends inside a respective cylindrical inner wall. The inner walls preferably define passages to receive the outlet tubes. The shape of the inner wall preferably correspond to the shape of the tube, which may be cylindrical as mentioned above. A passage is defined between the inner wall and the tubes, which allows the overflow of fluid and exposes the contents of the well to the atmosphere. The passage may extend coaxially around the tube between said tube and the inner wall.

Preferably, the fluid manifold is arranged to be supported onto, and to span, a plurality of wells, wherein the fluid manifold comprises outlet tubes for cooperation with only a part of said plurality of wells, wherein the fluid manifold further comprises an alignment protrusion to be received in at least one well not cooperating with an outlet tube. The alignment protrusion then ensures that the manifold is placed correctly, preferably in a way that the outlet tubes do not make contact with the inner walls of the respective wells. An empty well of the cell culture plate can be used for alignment.

A further preferred embodiment comprises a base part and a top part, wherein the base part comprises the overflow reservoir, wherein the base part is arranged to be supported onto the cell culture plate, wherein the top part comprises the outlet tubes and is arranged to be coupled onto the base part. The top and base part may then be removable coupled. The top part is preferably arranged at a distance from the bottom part, wherein the outlet tubes extend downwardly, preferably through the inner walls of the overflow reservoir.

A further preferred embodiment is manufactured from a material chosen from the group consisting of metal, preferably stainless steel, more preferable type 316L, and plastic, in particular PMMA, or a combination thereof.

According to a further aspect, an apparatus for culturing cells is provided, which is preferably arranged for performing the method as described above. The apparatus preferably comprises:
a holder for receiving a cell culture plate comprising at least one well;
a fluid manifold, preferably as defined above, wherein a fluid manifold fluid inlet is arranged to be received in a well of said cell culture plate and wherein a fluid manifold fluid inlet is arranged to receive fluid from said well;
a fluid output coupled to the fluid inlet of the fluid manifold;
a fluid container arranged for containing a second fluid, wherein the fluid container is coupled to the fluid outlet of the fluid manifold.

The apparatus is preferably provided with a fluid pumping system, for instance including a suitable controller, for replacing the fluid as mentioned above. The fluid pumping system is preferably arranged to replace a fluid in the well by removing the fluid from the well using the fluid manifold fluid inlet to the fluid output while introducing using the fluid manifold fluid outlet the second fluid from the fluid container in the well until the well is homogenously filled with the second fluid.

As mentioned above, the apparatus preferably further comprises a measuring unit arranged for performing measurement on the fluid, preferably coupled to the fluid manifold fluid inlet and arranged for analysing the fluid removed from at least one well.

The apparatus preferably further comprises a microscope arranged for analysing at least one cell held in the at least one well. The microscope may be provided with an image pickup device for obtaining images of the well. The microscope may comprise a bright field microscope, a fluorescence microscope and/or the microscope may rely on absorption microscopy techniques.

According to a further preferred embodiment, the holder is arranged to receive a cell culture plate comprising a plurality of wells, wherein the fluid manifold comprises a plurality of fluid outlets arranged to be received in a plurality of wells. The apparatus preferably comprises a plurality of manifolds, each arranged to be coupled to a plurality of wells. Each of manifolds is then preferably coupled to a fluid source for introduction of fluid in the respective wells.

The holder and any microscope, or other analysing device arranged to analyse the cell(s) in a well, are then preferably arranged movable with respect to each other. More preferably, the holder is arranged to move the wells plate relative to the microscope such that a plurality of wells can be analysed by the microscope.

A further preferred embodiment further comprises a cell culture plate comprising at least one well provided with at least one cell and a fluid, preferably a plurality of wells, each provided with at least one cell and a fluid, for instance cell culture medium.

The present invention is further illustrated by the following Figures, which show a preferred embodiment of the device according to the invention, and are not intended to limit the scope of the invention in any way, wherein:

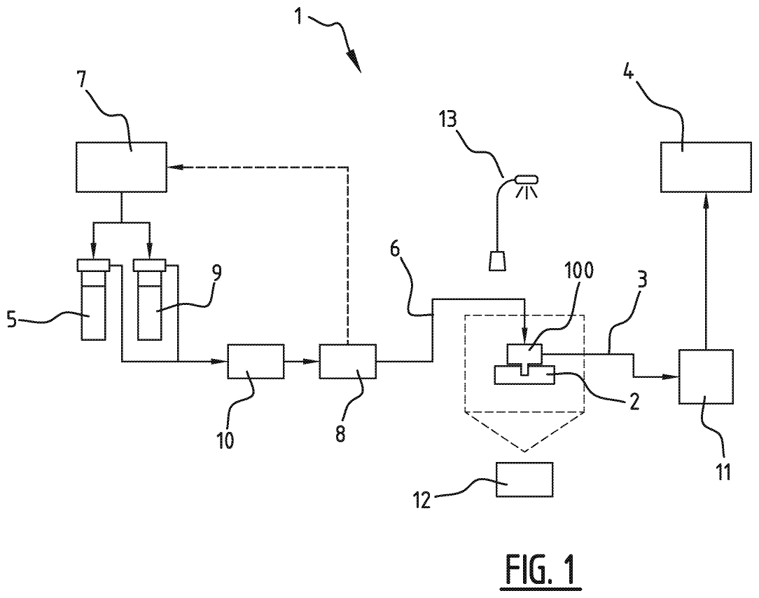

FIG. 1 schematically shows an apparatus for cultivating a cell culture in a cell culture plate;

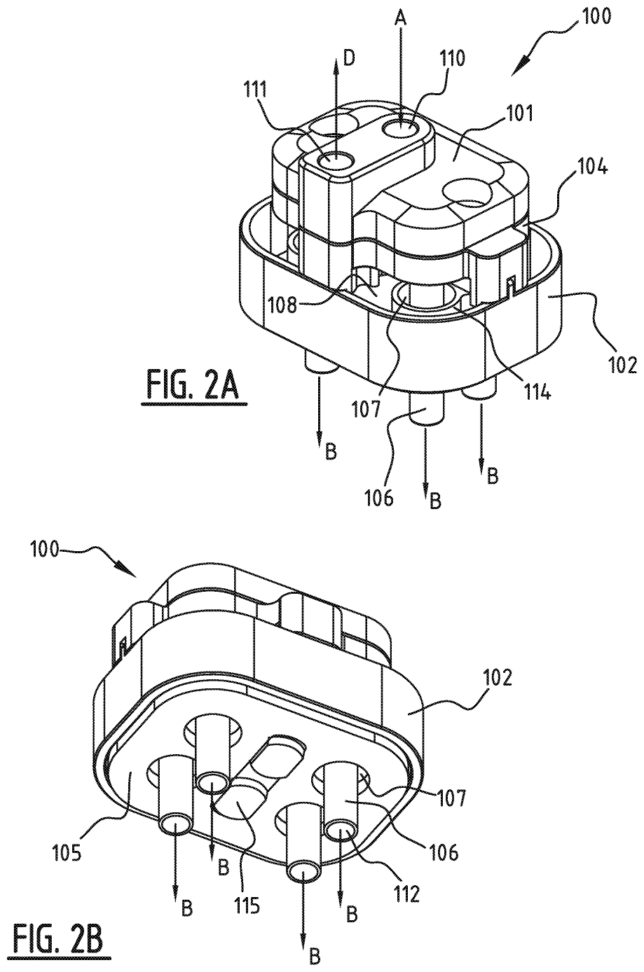

FIG. 2A shows a fluid manifold for use in the apparatus of FIG. 1 in a perspective view;

FIG. 2B shows the fluid manifold in a different perspective view;

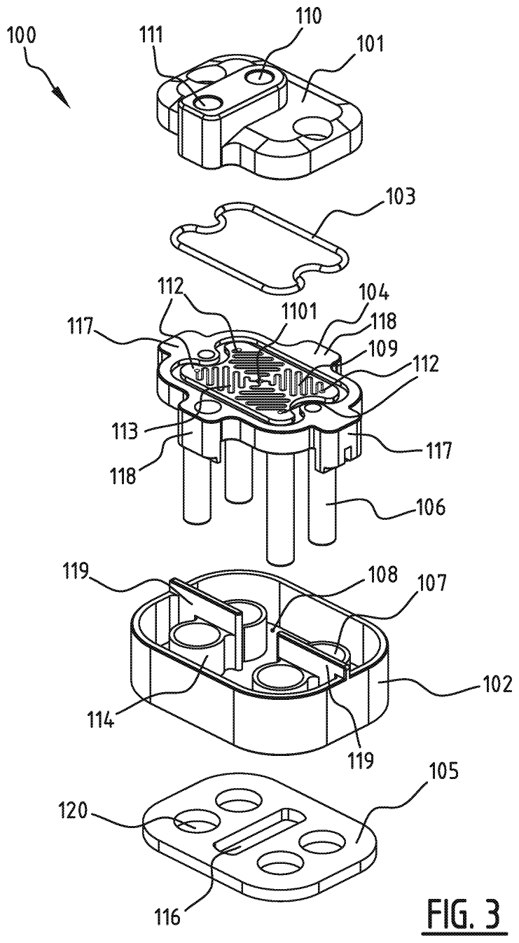

FIG. 3 shows an overview of the parts that make up the fluid manifold;

FIG. 4 shows a cross section of the fluid manifold;

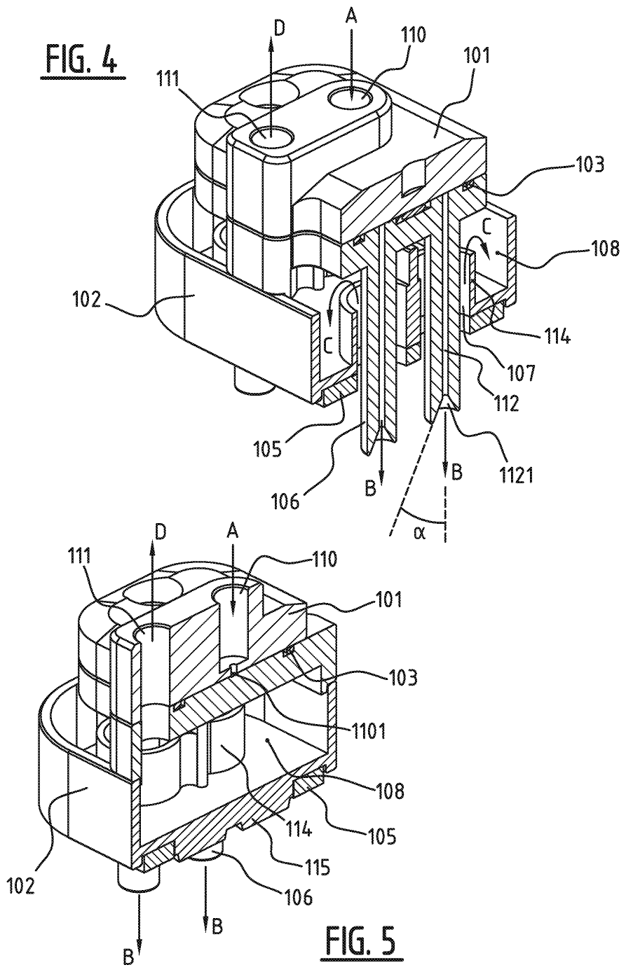

FIG. 5 shows a different cross section of the fluid manifold;

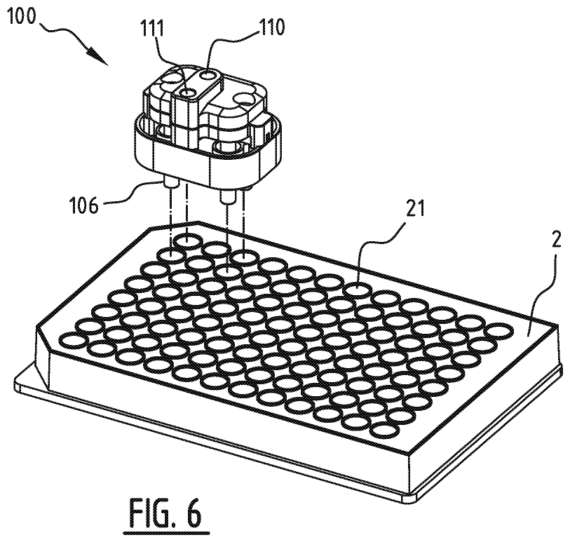

FIG. 6 shows the placement of the fluid manifold on a cell culture plate;

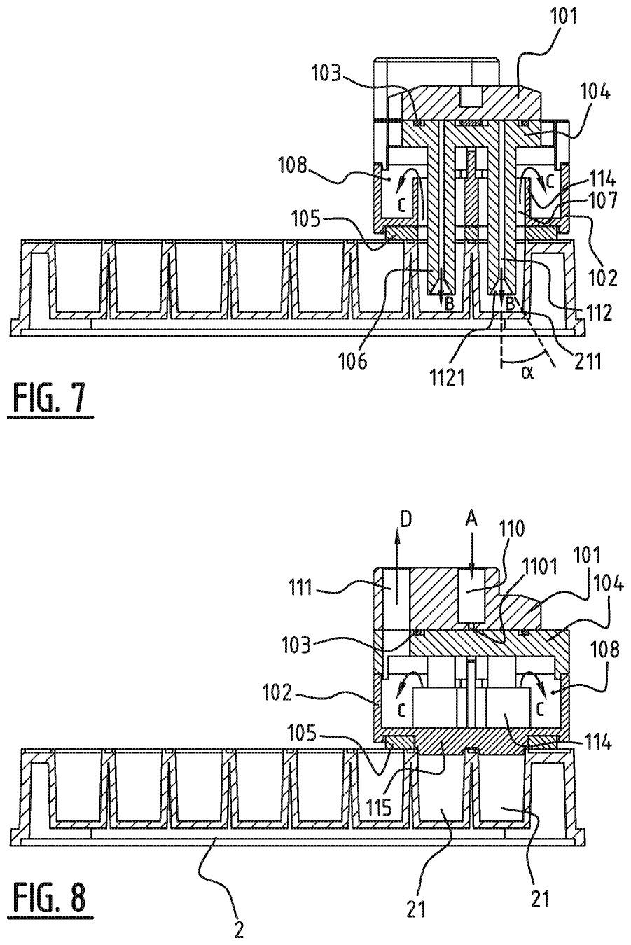

FIG. 7 shows a cross section of the fluid manifold interacting with a cell culture plate;

FIG. 8 shows a different cross section of the fluid manifold interacting with a cell culture plate, and;

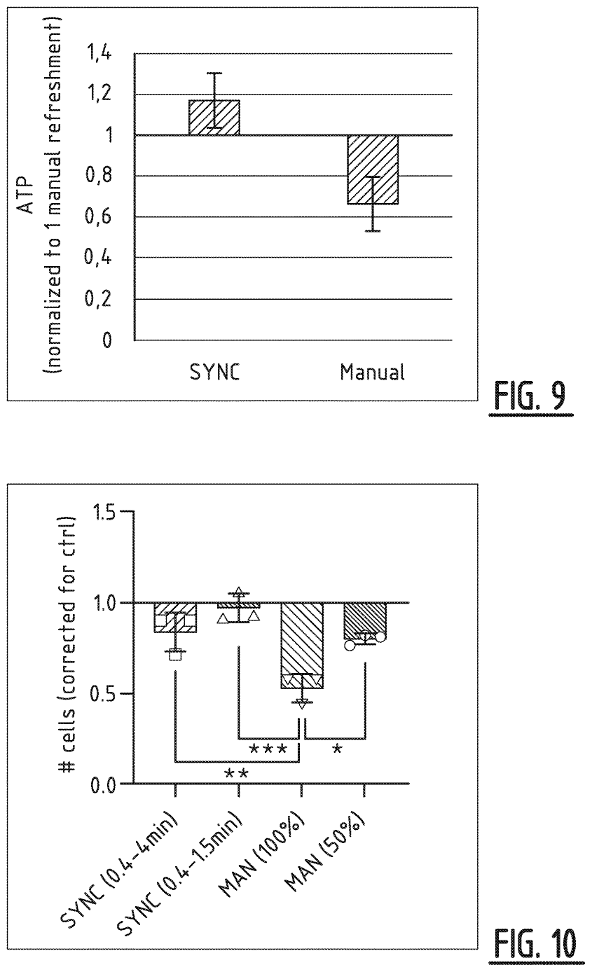

FIGS. 9 and 10 show results of experiments demonstrating the reduced impact on cells using the fluid manifold.

In FIG. 1, a schematic representation is shown of an apparatus 1 for cultivating a cell culture in a cell culture plate 2. The cell culture plate comprises a plurality of wells 21 (shown in FIG. 6) which contain at least a cell culture and a cell culture medium or first fluid. The first fluid may be removed from the one or more wells 21 and a second fluid may be introduced into the one or more wells 21 by means of a fluid manifold 100. The first fluid is removed from a well 21 by the second fluid being injected into the well 21. The first fluid is then pushed out of the well 21 into the fluid manifold 100 and subsequently removed from the fluid manifold 100 by means of a fluid connection 3 that connects through waste reservoir 11 to a vacuum source 4. The waste reservoir 11 stores the fluid removed from the wells 21 by the fluid manifold 100.

The second fluid is provided by the second fluid reservoir 5. The second fluid is transmitted from the second fluid reservoir 5 to the fluid manifold 100 through fluid connection 6 as a result of a pressure being applied to the second fluid reservoir 5 by the pressure controller 7. The flow rate of the second fluid from the second fluid reservoir 5 to the manifold 100 and subsequently to the wells 21 is controlled by controlling the pressure applied to the second fluid reservoir 5 by means of the pressure controller 7. The flow rate of the second fluid is measured by the flow sensor 8.

An optional third fluid reservoir 9 may be provided to supply a third cell culture medium or third fluid. A valve 10 is then employed to control whether the second fluid reservoir 5 and/or the third fluid reservoir 9 is connected to the fluid manifold 100 through the fluid connection 6. The third fluid reservoir 9 receives pressure from the pressure controller 7 in the same way as the second fluid reservoir 5. An optional measuring unit may be provided in the fluid connection 3 between the fluid manifold 100 and the waste reservoir 11 for analysing the fluid that is removed from the wells 21 of the cell culture plate 2. A microscope 12 may be provided to analyse the contents of the wells 21 of the cell culture plate 2, and a light source 13 may be provided.

Although not shown, the microscope 12 is moveable with respect to the cell culture plate 2 such that the microscope 12 can be aligned with preferably all wells 21 of the cell culture plate 2. Although the microscope 12 may be arranged movable, it is preferred if the cell culture plate 2, with manifolds 100, is provided movable, for instance on a scanning stage.

FIG. 2A shows a fluid manifold 100 for use in the apparatus of FIG. 1 in perspective view. This embodiment of the fluid manifold 100 comprises four outlet tubes 106 which extend into the wells 21 when the fluid manifold 100 is arranged on a cell culture plate 2. The fluid manifold 100 comprises a manifold outlet connection 110 for receiving the second or third fluid in flow direction A in the fluid manifold 100. The replacement fluid then flows through the fluid manifold 100 out of outlet tubes 106 in direction B for replacing the first fluid present in the wells 21 of the cell culture plate 2. A manifold inlet connection 111 is additionally provided for removing the waste fluid from the manifold in direction D.

FIG. 2B shows the fluid manifold 100 of FIG. 2A from a lower perspective. Lugs 115 are provided that interact with the top edges of two wells 21 laying between the two pairs of wells into which the outlet tubes 106 extend. The lugs 115 thereby locate the fluid manifold 100 at the appropriate location when placed on a cell culture plate 2.

FIG. 3 shows an overview of the parts that make up the fluid manifold 100. An upper part or centre manifold 104 is provided which distributes a flow of fluid from the outlet connection 110 to the four outlets tubes 106. To ensure that each outlet tube 106 outputs the fluid at the same and suitable low pressure in order not to disturb any cells in the wells, the manifold 100 comprises a flow restricting section comprising four flow restrictor channels 109, which connect to four fluid channels 112 arranged in the four outlet tubes 106. The centre manifold 104 further comprises two first alignment lugs 117 and two second alignment lugs 118.

A manifold cap 101 is provided to cover the top of the centre manifold 104, and a gasket 103, for instance made from a plastic or rubber, preferably nitrile butadiene rubber (NBR), is provided between the manifold cap 101 and the centre manifold 104 for ensuring a fluid tight connection. The manifold cap 101 covers the flow restrictor channels 109 forming flow restrictor tubes 109. The flow restrictor channels 109 connect to the manifold outlet connection 110 at centre point 1101. The fluid introduced into the fluid manifold 100 through fluid outlet connection 110 is equally divided across the flow restrictor tubes 109 ensuring an equal amount of fluid flows into the fluid channels 112 in the outlet tubes 106.

The manifold cap 101 further comprises a manifold inlet connection 111 for evacuating waste liquid from the fluid manifold 100. The centre manifold 104 is arranged in the reservoir section 102. The first alignment lugs 117 of the centre manifold 104 interact with the alignment members 119 and the top edge of the reservoir section 102, and the second alignment lugs 118 of the centre manifold 104 interact with the top edge of the reservoir section 102. A degree of interference may be provided in the interaction of alignment lugs 117, 118 and the reservoir section 102 so that the parts flexibly deform when put together which results in a clamping force which retains the centre manifold 102 in the reservoir section 104. Glue may additionally be used between the alignment lugs 117, 118 and the reservoir section 102 and alignment members 119, or between any other parts of the fluid manifold 100, to provide another retention means.

The reservoir section 104 further comprises an overflow reservoir 108 for receiving waste fluid, and four cylindrical sections 114 through which the outlet tubes 106 extend, defining four fluid inlets 107 for receiving the waste fluid from the wells 21.

The interaction of the alignment lugs 117, 118 with the reservoir section 104 and the alignment members 119 ensures that the centre manifold is located in the preferred position relative to the reservoir section. In this preferred position the outlet tubes 106 are centred in the cylindrical sections 114. Fluid inlets 107 or passages further expose an underlying well to the atmosphere outside the fluid manifold 100. A bottom gasket 105 is arranged on the underside of the reservoir section 102 for ensuring a fluid tight connection. Bottom gasket 105 comprises holes 120 through which the outlet tubes 106 extend and which extend the waste fluid inlets 107. Hole 116 is additionally provided through which lugs 115 extend.

FIG. 4 shows a cross section of the fluid manifold 100. Replacement fluid, such as the second and the third fluid, enters the fluid manifold 100 through manifold inlet connection 110 in direction A. The replacement fluid then flows through flow restrictor passages 109 (shown in FIG. 3) into the fluid channels 112 which extend through the outlet tubes 106 into the wells 21 in direction B. As a result of the injection of new fluid into wells 21, the old, waste fluid is pushed out of the wells 21 through inlets 107 into the overflow reservoir 108 in direction C. The waste fluid spills over the top edges of the four cylindrical sections 114 into the overflow reservoir 108. It is therefore not possible for waste fluid present in the overflow reservoir 108 to flow back into the wells 21 through the waste fluid inlets 107. The waste fluid is then removed from the overflow reservoir 108 through manifold inlet connection 111 in direction D. A tube member may be inserted in the fluid manifold 100 through the manifold inlet connection 111 which extends to below the waste fluid level present in the overflow reservoir 108 to extract the waste fluid from the overflow reservoir 108. The fluid level in the overflow reservoir 108 is preferably kept just below the upper edge of the cylindrical sections 114. Fluid channels 112 end with a widening section 1121. The fluid channels 112 extend along a centreline, and the walls of the widening section 1121 extend at an angle α relative to this centreline.

FIG. 5 shows a cross section of the fluid manifold 100 at a different place. Replacement fluid flows into the fluid manifold 100 through manifold outlet connection 110 in direction A. The four flow resistor channels 109 debouch into centre point 1101, through which the replacement fluid then flows to the four fluid channels 112 in the outlet tubes 106 (shown at least in FIG. 4) into the wells 21 in direction B. Lugs 115 are additionally shown.

FIG. 6 shows the placement of a single fluid manifold 100 on a cell culture plate 2. Four outlet tubes 106 extend into four corresponding wells 21. The four wells 21 receiving an outlet tube 106 can be divided into two sets of two. In between the first and second sets of two wells 21 is a third set of wells 21 that do not receive an outlet tube 106. Lugs 115 (shown in FIG. 2B) interact with this third set of wells 21 in order to locate the fluid manifold 100 at the appropriate location on the cell culture plate 2, and to retain the fluid manifold 100 in the horizontal plane. The present arrangement of the fluid manifold 100 allows it to be placed anywhere on the cell culture plate 2 in order to replace the cell culture fluid in any four wells 21. Typically, a plurality of manifolds 100 will be placed adjacently on the cell culture plate 2.

FIG. 7 shows a cross section showing the interaction of the fluid manifold 100 with a first or second set of wells 21. When the fluid manifold 100 is placed on the cell culture plate 2 the outlet tubes 106 extend into wells 21 which contain a cell culture and cell culture fluid to be replaced. As new culture fluid is injected into the wells 21 through fluid channels 112 arranged in the outlet tubes 106 in direction B, the old fluid is pushed out of the wells 21, thereby increasing the fluid level in the wells 21, pushing the fluid up through waste fluid inlets 107 and over the top edges of cylindrical sections 114 into the overflow reservoir 108 in direction C. Preferably, the walls of inlets 107 are aligned with the walls of the wells 21. The widening sections 1121 are also shown, in combination with the angle α of the walls of these widening sections 1121. Angle α is chosen such that the tangent of the walls of the widening sections 1121 point substantially in the direction of the bottom corners 211 of wells 21.

FIG. 8 shows a cross section at a different location showing the interaction of the fluid manifold 100 with the cell culture plate 2. Lugs 115 interact with the top edges of the third set of wells 21 which do not receive an outlet tube 106 that lay between the first and second set of wells 21 that do receive an outlet tube 106. Replacement fluid flows into the fluid manifold 100 through manifold outlet connector 110, through centre point 1101 and the four flow restrictor channels 109 (shown in FIG. 3) and fluid channels 112 (shown at least in FIG. 7) into the wells 21. Old fluid is pushed up and out of the wells 21 through inlets 107 and spills over the top edge of cylindrical sections 114 into the overflow reservoir 108 in direction C. Old fluid is then removed from the overflow reservoir 108 through manifold inlet connection 111 in direction D.

### Experiment 1

In order to determine how fast a fluid can be replaced in a single well using the manifold (as shown in FIGS. 2-8) and apparatus (see FIG. 1 for a schematic overview of the system used) of the invention, an experiment was conducted.

The two reservoirs 5 and 9 were filed with fluids having different colours (using food dyes). The flow rate was set at 1 ml/min and the fluid (colour) was changed 5 times. When a plateau of the colour intensity (see below) was reached, the valve 10 was operated to switch the supply of fluid to the manifold 100.

Video images were recorded and analysed at 5 locations (middle of well underneath nozzle, bottom right, left corner, top right and left corner). The experiment was performed in a dark environment, with a single light source at a controlled location. An average of the colour intensities measured at the five locations in the well was calculated and the time to reach 95% of the plateau (homogeneous replacement of the fluid in the well) was calculated. The results are listed in the table below:

| | Time to reach 95% of plateau [s] |
|---|---|
| Change 1 | 56.9 |
| Change 2 | 73.9 |
| Change 3 | 52 |
| Change 4 | 80.6 |
| Change 5 | 51 |

From the above, it follows that with the method, apparatus and manifold as disclosed, a quick and reliable replacement of fluid in a well can be achieved.

### Experiment 2

### Method

The day before the experiment, HepG2 cells were dissociated and seeded into uncoated 96 well plates at a density of 10,000 cells/well. On the day of the experiment, the cells underwent refreshment procedures, either through manual pipetting or using the fluid manifold (as shown in FIGS. 2-8) and the apparatus (see FIG. 1 for a schematic overview of the system used), designed to simulate the handling required to replicate a pharmacokinetic (PK) profile (i.e. introducing a new concentration of a specific drug every few minutes). In this case, the medium was fully replaced 6 times at 30-minute intervals, accomplished either by manual pipetting or using the fluid manifold set at a flow rate of 4 ml/minute for a duration of 1.5 min. Both methods were compared to a single manual replacement. After 24 hours, cell viability was assessed by quantifying ATP levels using a Cell Titer Glo luminescence assay (Promega) per manufacturer's protocol. Each condition was tested using 4 wells, and the experiment was repeated three times (N=3).

### Outcome

With reference to FIG. 9, it can be seen that the fluid manifold has less impact on cell attachment compared to manual pipetting. Performing multiple replacement steps, six in this instance, such as those required for simulating a pharmacokinetic profile of a specific drug, does not result in a decrease of viable cells when utilizing the fluid manifold. In contrast, manually pipetting such a replacement schedule leads to a reduction of over 33% in viable cells. This indicates that the fluid manifold outperforms manual pipetting in multiple fluid replacements with respect to maintaining cell attachment and viability.

### Experiment 3

### Method

On the day of the experiment, HepG2 cells were dissociated and plated into 96-well plates pre-coated with an anti-adherence solution to keep the cells in suspension and avoid cell adherence (20,000 cells/well). 15 minutes post-plating (based on the sediment time determined by microscopy), the experiment was performed. 100% or 50% of the medium was refreshed manually or by using the fluid manifold of this disclosure using a flow rate of 0.4 ml/min for 4 minutes (simulating a 100% refreshment) or 0.4 ml/min for 1.5 minute (simulating an at least 50% refreshment). After the refreshment step, cells were collected and manually counted. The number of cells left after 1 refreshment step was corrected for cells left without a refreshment step. Each condition was tested using 4 wells, and the experiment was repeated 3 times (N=3). Significance was determined after performing one-way ANOVA. *P<0.05, **P<0.01, ***P<0.001

### Outcome

Using the fluid manifold of the present disclosure, medium of suspension cells can be refreshed under flow while keeping cell loss to a minimum. The fluid manifold minimizes cell loss in suspension culture during media refreshments, see FIG. 10. The “SYNC (0.4-4 min)” was comparable in terms of cell loss with the “MAN-50%” condition and greatly outperformed the “MAN-100%”. The “SYNC (0.4-1.5 min)” seemed to also outperform the “MAN-50%” condition although this was not statistically significant. We are confident that by tuning the flow rate and flow time we can alter the total percentage media being replaced as desired, also for other suspension cells.

The present invention is further illustrated by the following embodiments:
1. Method for cultivating at least one cell in a cell culture plate, wherein the method comprises the steps of:
   providing a cell culture plate comprising at least one well containing at least one cell and a first fluid, for instance a cell culture medium;
   providing a second fluid, for instance a cell culture medium, in a fluid container;
   providing a fluid manifold comprising a fluid outlet extending in the well and coupled to the fluid container and a fluid inlet arranged to receive fluid from said well and coupled to an fluid output;
   replacing the fluid in said well by introducing, from said fluid outlet, said second fluid in said well while removing, using said fluid inlet, fluid from said well until the well is homogenously filled with the second fluid.
2. Method according to embodiment 1, wherein the step of replacing the fluid comprises maintaining a substantial constant fluid level in said well.
3. Method according to embodiment 1 or 2, wherein the step of introducing the second fluid comprising introducing said second fluid at a sufficiently low flow rate and/or pressure to prevent disturbing the at least one cell.
4. Method according to embodiment 3, wherein the step of introducing the second fluid comprising introducing said second fluid at a sufficiently low flow rate and/or pressure to exert a shear stress on said at least one cell lower than 0.1 Pa, preferably lower than 0.05 Pa, more preferably lower than 0.01 Pa.
5. Method according to any of the preceding embodiments, wherein the step of introducing the second fluid comprising introducing said second fluid at a flow rate of between 0.2 and 1.4 ml/min, preferably between 0.8 and 1.2 ml/min, more preferably between 0.95 and 1.05 ml/min.
6. Method according to any of the preceding embodiments, wherein the step of replacing the fluid takes within 5 minutes, preferably within 1.5 minutes, more preferably within 1 minute.
7. Method according to any of the preceding embodiments, wherein the step of introducing the second fluid comprises introducing said second fluid from a nozzle of said outlet from a location below the fluid level in said well.
8. Method according to embodiment 7, wherein the second fluid is introduced from said nozzle extending at a distance of 0.1-5 mm, preferably between 1-4 mm, more preferably between 2-3 mm, from a bottom of said well.
9. Method according to embodiment 7 or 8, wherein the nozzle comprises a fluid channel widening towards the outlet side of the outlet.
10. Method according to embodiment 9, wherein the wall of the widening channel is substantially aligned with the corner between the bottom and the side wall of the well.
11. Method according to any of the preceding embodiments, wherein the step of replacing the fluid comprises introducing the second fluid in the in well and removing the fluid at a second location, not in fluid communication with the well.
12. Method according to any of the preceding embodiments, wherein the step of removing fluid from the well comprises receiving overflow fluid from said well in an overflow reservoir and removing, using said fluid inlet, fluid from said overflow reservoir.
13. Method according to any of the preceding embodiments, further comprising the steps of providing a third fluid and replacing the second fluid in said well by introducing, from said fluid outlet, said third fluid in said well while removing, using said fluid inlet, fluid from said well until the well is homogenously filled with the third fluid.
14. Method according to any of the preceding embodiments for cultivating a plurality of cells in a plurality of wells in a cell culture plate, wherein:
   the step of providing the cell culture plate comprises providing a cell culture plate comprising a plurality of wells, wherein a plurality of said wells contain at least one cell and a fluid such as a cell culture medium;
   the step of providing the fluid manifold comprises providing a fluid manifold comprising a plurality of fluid outlets extending in respective wells and coupled to a fluid container containing the second fluid;
   replacing the fluid in at least one of said wells by introducing, from at least one fluid outlet, said second fluid in said well while removing, using said fluid inlet, fluid from said well until the well is homogenously filled with the second fluid.
15. Method according to embodiment 14, wherein the step of replacing the fluid comprises introducing, from a plurality of fluid outlets, said second fluid in a plurality of wells while removing, using said fluid inlet, fluid from said wells until the wells are homogenously filled with the second fluid.
16. Method according to embodiment 14 or 15, wherein the step of removing fluid from the well comprises receiving overflow fluid from a plurality of wells in an overflow reservoir and removing, using said fluid inlet, fluid from said overflow reservoir.
17. Method according to any of the preceding embodiments, further comprising the step of analysing the at least one cell using a microscope.
18. Method according to any of the preceding claims 1-17, further comprising the step of analysing the fluid from the well received in the fluid output.
19. Fluid manifold for use in a method according to any of the preceding embodiments, wherein the manifold is arranged to be coupled to a cell culture well, for instance of a cell culture plate comprising a plurality of cell culture wells, wherein the fluid manifold comprises:
   a fluid outlet comprising an outlet tube arranged to introduce fluid in a well at a first location, wherein the fluid outlet is coupled to a fluid outlet connector;
   a fluid inlet arranged to receive fluid from said well at a second location, wherein said fluid inlet is coupled to a fluid inlet connector,
   wherein the fluid manifold is arranged to couple to said well such that said first location of introducing the fluid is inside said well and said second location for receiving the fluid is outside the well, wherein in use the first and second locations are not in fluid communication.
20. Fluid manifold according to embodiment 19, wherein the outlet tube comprises a nozzle formed by a fluid channel widening towards the outlet side of the outlet tube.
21. Fluid manifold according to embodiment 20, wherein the fluid manifold is arranged to couple to said well such that the wall of the widening channel is substantially aligned with the corner between the bottom and the side wall of the well.
22. Fluid manifold according to any of the preceding embodiments, wherein the manifold is arranged to be coupled to a plurality of wells, wherein the manifold comprises a plurality of outlet tubes, each arranged for introducing fluid in a respective well, wherein the plurality of outlet tubes are coupled to a common fluid outlet connector.
23. Fluid manifold according to embodiment 22, wherein the plurality of outlet tubes are coupled to the common fluid outlet connector using respective flow resistors.
24. Fluid manifold according to any of the preceding embodiments, further comprising an overflow reservoir for receiving fluid from said well, wherein the fluid inlet is arranged to remove fluid from the overflow reservoir.
25. Fluid manifold according to embodiment 24, wherein the outlet tube is arranged for introducing fluid from a lower outlet side thereof, wherein the overflow reservoir extends at a location above the lower outlet side of the outlet tube.
26. Fluid manifold according to embodiment 24 or 25, wherein the overflow reservoir comprises an outer wall and a cylindrical inner wall, defining the reservoir therebetween, wherein the outlet tube of the outlet extends inside the cylindrical inner wall at a distance from said inner wall for defining a passage for fluid from the well into the overflow reservoir.
27. Fluid manifold according to embodiment 26, wherein the fluid reservoir and the passage for fluid are open to the environment of the fluid manifold.
28. Fluid manifold according to at least embodiments 22 and 26, wherein the overflow reservoir comprises a plurality of cylindrical inner walls, wherein each of the plurality of outlet tubes extends inside a respective cylindrical inner wall.
29. Fluid manifold according to embodiment 28, wherein the fluid manifold is arranged to be supported onto, and to span, a plurality of wells, wherein the fluid manifold comprises outlet tubes for cooperation with only a part of said plurality of wells, wherein the fluid manifold further comprises an alignment protrusion to be received in at least one well not cooperating with an outlet tube.
30. Fluid manifold according to any of the preceding embodiment 27 or 28, comprising a base part and a top part, wherein the base part comprises the overflow reservoir, wherein the base part is arranged to be supported onto the cell culture plate, wherein the top part comprises the outlet tubes and is arranged to be coupled onto the base part.
31. Fluid manifold according to any of the preceding embodiments, manufactured from a material chosen from the group consisting of metal, preferably stainless steel, more preferable type 316L, and plastic, in particular PMMA, or a combination thereof.
32. Apparatus for culturing cells arranged for performing the method according to any of the preceding embodiments 1-18 comprising:
   a holder for receiving a cell culture plate comprising at least one well;
   a fluid manifold according to any of the preceding embodiments, wherein a fluid manifold fluid inlet is arranged to be received in a well of said cell culture plate and wherein a fluid manifold fluid inlet is arranged to receive fluid from said well;
   a fluid output coupled to the fluid inlet of the fluid manifold;
   a fluid container arranged for containing a second fluid, wherein the fluid container is coupled to the fluid outlet of the fluid manifold;
   a fluid pumping system arranged to replace a fluid in the well by removing the fluid from the well using the fluid manifold fluid inlet to the fluid output while introducing using the fluid manifold fluid outlet the second fluid from the fluid container in the well until the well is homogenously filled with the second fluid.
33. Apparatus for culturing cells according to embodiment 32, further comprising a measuring unit coupled to the fluid manifold fluid inlet and arranged for analysing the fluid removed from at least one well.
34. Apparatus for culturing cells according to embodiment 32 or 33, further comprising a microscope arranged for analysing the at least one cell held in the at least one well.
35. Apparatus according to embodiment 32, 33 or 34, wherein the holder is arranged to receive a cell culture plate comprising a plurality of wells, wherein the fluid manifold comprises a plurality of fluid outlets arranged to be received in a plurality of wells.
36. Apparatus according to embodiment 35, comprising a plurality of manifolds, each arranged to be coupled to a plurality of wells.
37. Apparatus according to at least embodiments 34 and 35, wherein the holder is arranged to move the wells plate relative to the microscope such that a plurality of wells can be analysed by the microscope.
38. Apparatus according to any of the preceding embodiments, further comprising a cell culture plate comprising at least one well provided with at least one cell and a fluid, preferably a plurality of wells, each provided with at least one cell and a fluid, for instance cell culture medium.

The present invention is not limited to the embodiment listed or shown, but extends also to other embodiments falling within the scope of the appended claims.

1-40. (canceled)

41. A method for cultivating at least one cell in a cell culture plate, wherein the method comprises the steps of:
providing a cell culture plate comprising at least one well containing at least one cell and a first fluid, for instance a cell culture medium;
providing a second fluid, for instance a cell culture medium, in a fluid container;
providing a fluid manifold comprising a fluid outlet extending in the well and coupled to the fluid container and a fluid inlet arranged to receive fluid from said well and coupled to an fluid output; and
replacing the fluid in said well by introducing, from said fluid outlet, said second fluid in said well while removing, using said fluid inlet, fluid from said well until the well is homogenously filled with the second fluid.

42. The method according to claim 41, wherein the step of replacing the fluid comprises maintaining a substantial constant fluid level in said well.

43. The method according to claim 41, wherein the step of introducing the second fluid comprises introducing said second fluid at a sufficiently low flow rate and/or pressure to exert a shear stress on said at least one cell lower than 0.1 Pa, preferably lower than 0.05 Pa, more preferably lower than 0.01 Pa.

44. The method according to claim 41, wherein the step of introducing the second fluid comprising introducing said second fluid at a flow rate of between 0.2 and 1.4 ml/min, preferably between 0.8 and 1.2 ml/min, more preferably between 0.95 and 1.05 ml/min, wherein the step of replacing the fluid takes within 5 minutes, preferably within 1.5 minutes, more preferably within 1 minute.

45. The method according to claim 41, wherein the step of introducing the second fluid comprises introducing said second fluid from a nozzle of said outlet from a location below the fluid level in said well.

46. The method according to claim 45, wherein the second fluid is introduced from said nozzle extending at a distance of 0.1-5 mm, preferably between 1-4 mm, more preferably between 2-3 mm, from a bottom of said well.

47. The method according to claim 46, wherein the nozzle comprises a fluid channel widening towards the outlet side of the outlet, wherein the wall of the widening channel is substantially aligned with the corner between the bottom and the side wall of the well.

48. The method according to claim 41, wherein the step of replacing the fluid comprises introducing the second fluid in the in well and removing the fluid at a second location, not in fluid communication with the well.

49. The method according to claim 41, wherein the step of removing fluid from the well comprises receiving overflow fluid from said well in an overflow reservoir and removing, using said fluid inlet, fluid from said overflow reservoir.

50. The method according to claim 41 for cultivating a plurality of cells in a plurality of wells in a cell culture plate, wherein:
the step of providing the cell culture plate comprises providing a cell culture plate comprising a plurality of wells, wherein a plurality of said wells contain at least one cell and a fluid such as a cell culture medium;
the step of providing the fluid manifold comprises providing a fluid manifold comprising a plurality of fluid outlets extending in respective wells and coupled to a fluid container containing the second fluid; and
replacing the fluid in at least one of said wells by introducing, from at least one fluid outlet, said second fluid in said well while removing, using said fluid inlet, fluid from said well until the well is homogenously filled with the second fluid;
wherein the step of replacing the fluid comprises introducing, from a plurality of fluid outlets, said second fluid in a plurality of wells while removing, using said fluid inlet, fluid from said wells until the wells are homogenously filled with the second fluid.

51. The method according to claim 50, wherein the step of removing fluid from the well comprises receiving overflow fluid from a plurality of wells in an overflow reservoir and removing, using said fluid inlet, fluid from said overflow reservoir.

52. A fluid manifold for use in a method according to claim 41, wherein the manifold is arranged to be coupled to a cell culture well, for instance of a cell culture plate comprising a plurality of cell culture wells, wherein the fluid manifold comprises:
a fluid outlet comprising an outlet tube arranged to introduce fluid in a well at a first location from a lower outlet side thereof, wherein the fluid outlet is coupled to a fluid outlet connector; and
a fluid inlet arranged to receive fluid from said well at a second location, wherein said fluid inlet is coupled to a fluid inlet connector.

53. The fluid manifold according to claim 52, further comprising an overflow reservoir for receiving fluid from said well, wherein the fluid inlet is arranged to remove fluid from the overflow reservoir, wherein the overflow reservoir extends at a location above the lower outlet side of the outlet tube.

54. The fluid manifold according to claim 52, wherein the manifold is arranged to be coupled to a plurality of wells, wherein the manifold comprises a plurality of outlet tubes, each arranged for introducing fluid in a respective well, wherein the plurality of outlet tubes are coupled to a common fluid outlet connector, wherein the plurality of outlet tubes are coupled to the common fluid outlet connector using respective flow resistors.

55. The fluid manifold according to claim 53, wherein the overflow reservoir comprises an outer wall and a cylindrical inner wall, defining the reservoir therebetween, wherein the outlet tube of the outlet extends inside the cylindrical inner wall at a distance from said inner wall for defining a passage for fluid from the well into the overflow reservoir.

56. The fluid manifold according to claim 55, wherein the outlet tube is centred in the cylindrical inner wall, wherein the cylindrical inner wall is aligned with the wall of a well, and wherein the outlet tube is arranged to be centred in the well when the manifold is placed on a wells plate, wherein the fluid reservoir and the passage for fluid are open to the environment of the fluid manifold, wherein the overflow reservoir comprises a plurality of cylindrical inner walls, wherein each of the plurality of outlet tubes extends inside a respective cylindrical inner wall.

57. The fluid manifold according to claim 56, wherein the fluid manifold is arranged to be supported onto, and to span, a plurality of wells, wherein the fluid manifold comprises outlet tubes for cooperation with only a part of said plurality of wells, wherein the fluid manifold further comprises an alignment protrusion to be received in at least one well not cooperating with an outlet tube, wherein the fluid manifold further comprises a base part and a top part, wherein the base part comprises the overflow reservoir, wherein the base part is arranged to be supported onto the cell culture plate, wherein the top part comprises the outlet tubes and is arranged to be coupled onto the base part.

58. An apparatus for culturing cells arranged for performing the method according to claim 41 comprising:
a holder for receiving a cell culture plate comprising at least one well;
a fluid manifold, wherein a fluid manifold fluid inlet is arranged to be received in a well of said cell culture plate and wherein a fluid manifold fluid inlet is arranged to receive fluid from said well;
a fluid output coupled to the fluid inlet of the fluid manifold;
a fluid container arranged for containing a second fluid, wherein the fluid container is coupled to the fluid outlet of the fluid manifold; and
a fluid pumping system arranged to replace a fluid in the well by removing the fluid from the well using the fluid manifold fluid inlet to the fluid output while introducing using the fluid manifold fluid outlet the second fluid from the fluid container in the well until the well is homogenously filled with the second fluid.

59. The apparatus for culturing cells according to claim 58, further comprising a measuring unit coupled to the fluid manifold fluid inlet and arranged for analysing the fluid removed from at least one well.

60. The apparatus for culturing cells according to claim 58, further comprising a microscope arranged for analysing the at least one cell held in the at least one well, wherein the holder is arranged to receive a cell culture plate comprising a plurality of wells, wherein the fluid manifold comprises a plurality of fluid outlets arranged to be received in a plurality of wells, wherein the holder is arranged to move the wells plate relative to the microscope such that a plurality of wells can be analysed by the microscope.
